(19) United States
(12) Patent Application Publication
(10) Pub. No.: US 2024/0180494 A1
(43) Pub. Date: Jun. 6, 2024

(54) Signal processing method and apparatus

(71) Applicant: BEIJING TSINGRAY TECHNOLOGY CO., LTD., Beijing (CN)

(72) Inventors: Yuguo DING, Beijing (CN); Wenyu ZHANG, Beijing (CN)

(21) Appl. No.: 17/787,598

(22) PCT Filed: May 27, 2021
(86) PCT No.: PCT/CN2021/096538

§ 371 Date: Jun. 21, 2022

(51) Int. Cl. A61B 5/00; A61B 5/0205; A61B 5/024; A61B 5/05; A61B 5/08; A61B 5/113; G16H 50/70

(52) U.S. Cl. CPC A61B 5/7207; A61B 5/0205; A61B 5/05; A61B 5/1135; A61B 5/7225; A61B 5/7257; G16H 50/70; A61B 5/02444; A61B 5/0816

(57) ABSTRACT

The present disclosure provides a signal processing method and apparatus. The method includes: extracting the chest cavity movement signal from the radar echoes, which are the electromagnetic signals reflected from the target; extracting the respiratory signal and heartbeat signal by performing the bandpass filters on the chest cavity movement signal, wherein the respiratory signal corresponds to a respiratory bandpass frequency of the filter, and the heartbeat signal corresponds to a heartbeat bandpass frequency of the filter; performing joint time-frequency analysis on the respiratory signal to obtain a respiratory frequency, and performing joint time-frequency analysis and statistical analysis on the heartbeat signal to obtain the heartbeat frequency corresponding to the heartbeat signal.

### TECHNICAL FIELD

The present disclosure relates to the field of radar identification, and specifically, to a signal processing method and apparatus.

### BACKGROUND

The radar radiates electromagnetic signals to a target surface and receives echoes. The delay of the echo represents the distance between the target and the radar. According to the type of transmitted signals, the radar system can be divided into pulse radar and continuous wave radar. Traditional pulse radar emits periodic high-frequency pulses, while continuous wave radar emits continuous wave signals. Continuous wave radar can be used for range and speed measurements by emitting a frequency-modulated continuous wave signal, which is a continuous wave with varying frequencies within a sweep frequency period. There is a certain difference in frequency between the echo reflected by the target and a transmitted signal, which can be utilized to extract the distance information between the target and the radar.

At present, most of the devices utilized for monitoring of respiration and heartbeat are wearable devices. These devices require direct contact with the chest cavity or the pulse of the human body, which is not convenient and comfortable in many situations and has a great impact on human's work and rest. Therefore, the non-contact monitoring approaches and devices of respiration and heartbeat are desirable.

During the respiration of the human body, the chest cavity rises and falls with respiration. During the beating of the heart, the pumping action of the heart also causes the chest cavity to vibrate, although the vibration of the chest cavity caused by the heartbeat is very weak. Therefore, it is very important for the measurement of respiration and heartbeats to accurately detect the rise and fall of the chest cavity. In addition, it is particularly important to be able to accurately extract the signals of respiration and heartbeats from the movements of the chest cavity.

In the related art, Doppler radar is used to detect the respiratory frequency and heartbeat frequency of the human body. In this method, the respiratory frequency and heartbeat frequency are estimated by counting the number of peaks in the respiratory signal and the heartbeat signal in time domain, respectively. The main disadvantages of the method are that: the estimation results, which are completely dependent on the waveforms of respiration signal and heartbeat signal in time domain, can be easily interfered by noise. Moreover, this method requires high-quality signals, which increases the hardware cost. Furthermore, the frequency resolution of this method is insufficient.

In the related art, a remote sensing system of visible light may further be used to detect respiratory frequency and heartbeat frequency of the human body. In this method, the information of the movements of the chest cavity is collected by irradiating the visible light to the human chest cavity, and the respiratory frequency and heartbeat frequency are estimated from the spectrum in frequency domain with the help of the bandpass filters. The main disadvantages of the method are that the frequency of heartbeat is only estimated in the frequency domain, which is susceptible to noise and clutter.

In the related art, the heartbeat signal is extracted from the radar echoes under a strong noise condition. Firstly, the radar echo is transmitted to a data preprocessing terminal in the form of data frames to obtain the range unit of the human body, and a filter is utilized to remove the stationary background. In order to reduce the estimation error, the human movements are determined based on the acceleration of the human body. Then, the respiratory signal and the heartbeat signal are separated by an adaptive wavelet scale selection algorithm. Subsequently, the respiratory frequency and the heartbeat frequency are obtained by performing peak-searching process and down sampling process on the respiratory signal and the heartbeat signal, respectively. However, this method, which only focuses on the information in time domain of the signal and ignores information in the frequency-domain, can be easily affected by noise, while the noise can bring in extra peaks and valleys in the signal (that is, burr phenomenon).

In view of the above problems, no effective solution has been proposed yet.

### SUMMARY

Embodiments of the present disclosure provide a signal processing method and apparatus to solve the technical problem of low estimation accuracy of respiration rate and heartbeat rate, which is induced by utilizing the information from only the time domain or frequency domain.

According to an aspect of an embodiment of the present disclosure, a signal processing method is provided and includes: extracting the chest cavity movement signal from the radar echoes, which are the electromagnetic signals reflected from the target; extracting the respiratory signal and heartbeat signal by performing the bandpass filters on the chest cavity movement signal, wherein the respiratory signal corresponds to a respiratory bandpass frequency of the filter, and the heartbeat signal corresponds to a heartbeat bandpass frequency of the filter; performing joint time-frequency analysis on the respiratory signal to obtain a respiratory frequency, and performing joint time-frequency analysis and statistical analysis on the heartbeat signal to obtain the heartbeat frequency corresponding to the heartbeat signal.

In some embodiments of the present disclosure, the step of extracting the chest cavity movement signal from radar echoes includes: performing Fourier transform on each modulation signal from the radar echoes along time axis to obtain the range profiles; extracting the signals from the range bins with a maximum magnitude along the range axis in the range profiles and arranging the signals according to the time axis to obtain the respiratory-heartbeat signal; and performing unwrapping process on the respiratory-heartbeat signal to obtain the chest cavity movement signal.

In some embodiments of the present disclosure, the step of extracting the respiratory signal and heartbeat signal by performing bandpass filters on the chest cavity movement signal includes: setting the passband of the filter as the respiratory bandpass frequency, and performing the filter on the chest cavity movement signal to obtain the respiratory signal; and setting the passband of the filter as the heartbeat bandpass frequency, and performing the filter on the chest cavity movement signal to obtain the heartbeat signal.

In some embodiments of the present disclosure, the step of performing joint time-frequency processing on the respiratory signal to obtain the respiratory frequency includes: determining the effective number of respiratory periods of the respiratory signal in time domain; determining the effective respiratory frequency estimated in time domain according to the effective number of respiratory periods and the duration of the respiratory signal; performing Fourier transform on the respiratory signal to obtain the frequency spectrum in frequency domain; and determining the respiratory frequency corresponding to the respiratory signal according to the effective respiratory frequency estimated in time domain and the frequency spectrum of the respiratory signal in frequency domain.

In some embodiments of the present disclosure, the step of determining the effective number of respiratory periods of the respiratory signal in time domain includes: obtaining the first peak position sequence, that is, the position of peaks with the value is greater than 0, and the first valley position sequence, that is, the position of valleys with the value is less than 0, by traversing the respiration signal in time domain; eliminating peaks of which distance is less than the first preset threshold from the first peak position sequence and setting the number of remaining peaks as the number of first effective peaks, and eliminating valleys of which distance is less than the first preset threshold from the first valley position sequence and setting the number of remaining valleys as the number of first effective valleys; and determining the effective number of respiratory periods according to the number of first effective peaks and the number of first effective valleys.

In some embodiments of the present disclosure, the step of determining the respiratory frequency corresponding to the respiratory signal according to the effective respiratory frequency estimated in time domain and the frequency spectrum of the respiratory signal in frequency domain includes: determining the effective respiratory frequency range according to the effective respiratory frequency estimated in time domain and the allowable respiratory frequency value; determining the frequency with the maximum magnitude within the effective respiratory frequency range from the spectrum of the respiratory in frequency domain as the respiratory frequency.

In some embodiments of the present disclosure, the step of performing joint time-frequency analysis and statistical analysis on the heartbeat signal to obtain the heartbeat frequency corresponding to the heartbeat signal includes: determining the effective number of heartbeat periods of the heartbeat signal in time domain; estimating the effective frequency of the heartbeat signal in time domain according to the effective number of heartbeat periods and the duration of the heartbeat signal; performing Fourier transform on the heartbeat signal to obtain the spectrum of the heartbeat signal in frequency domain; and estimating the heartbeat frequency of the heartbeat signal according to the effective frequency estimated in time domain and the spectrum of the heartbeat signal in frequency domain.

In some embodiments of the present disclosure, before determining the effective number of heartbeat periods of the heartbeat signal in time domain, the method further includes: performing normalization process on the heartbeat signal to normalize the variance of the heartbeat signal; and determining the effective number of heartbeat periods of the heartbeat signal in time domain according to the normalized heartbeat signal.

In some embodiments of the present disclosure, the step of determining the effective number of heartbeat periods of the heartbeat signal in time domain includes: obtaining the second peak position sequence and the second valley position sequence, wherein the second peak position sequence is the sequence of the positions of peaks greater than 0, and the second valley position sequence is the sequence of the positions of valleys less than 0, respectively, by traversing the heartbeat signal; eliminating the peaks of which distance is less than the second preset threshold from the second peak position sequence and defining the number of remaining peaks as the number of second effective peaks, and eliminating the valleys of which distance is less than the second preset threshold from the second valley position sequence and defining the number of remaining valleys as the number of second effective valleys; and determining the effectively number of heartbeat periods according to the number of second effective peaks and the number of second effective valleys.

In some embodiments of the present disclosure, the step of determining the heartbeat frequency corresponding to the heartbeat signal according to the effective frequency of the heartbeat signal in time domain and the spectrum distribution of the heartbeat signal includes: determining the effective heartbeat frequency range according to the effective frequency of the heartbeat signal in time domain and the allowable heartbeat frequency value; extracting all the peaks from the spectrum distribution of the heartbeat signal within the interval of the effective heartbeat frequency range, and defining these peaks as heartbeat frequency candidates; extracting reserved heartbeat frequency set from the heartbeat frequency candidates; performing maximum likelihood estimation (MLE) on the reserved heartbeat frequency set to determine the probabilities of being the heartbeat frequency for all the frequencies in the reserved heartbeat frequency set; modifying the probability of the frequencies in the reserved heartbeat frequency set of being the heartbeat frequency via the analysis of historical data, and determining the frequency in the reserved heartbeat frequency with the maximum probability as the heartbeat frequency.

In some embodiments of the present disclosure, the step of performing MLE on the reserved heartbeat frequency set to determine the probabilities of being the heartbeat frequency for all the frequencies in the reserved heartbeat frequency set includes: determining the joint probability density function of the heartbeat signal according to the signal model of the heartbeat signal; determining a optimization problem of the parameters including the frequency, the amplitude and the initial phase of the heartbeat signal according to the joint probability density function; obtaining the probability of each frequency in the reserved heartbeat frequency set of being the real heartbeat frequency by solving the optimization problem.

In some embodiments of the present disclosure, the step of modifying the probability of the frequency in the reserved heartbeat frequency set of being the real heartbeat frequency via the analysis of historical data, and determining the frequency in the reserved heartbeat frequency set with the maximum probability as the heartbeat frequency includes: determining the historical heartbeat frequencies, which are the estimation results of the heartbeat signal in the past within the first preset time; performing cluster process on the historical heartbeat frequencies while taking each frequency in the reserved heartbeat frequency set as the centroid, and determining the ratio of the number of the historical heartbeat frequencies that are clustered as the class of each centroid (the frequency in the reserved heartbeat frequency set) to the total number; modifying the probability of the frequency in the reserved heartbeat frequency set of being the real heartbeat frequency by multiplying the ratio obtained from the cluster process; and taking the frequency in the reserved heartbeat frequency set with the maximum probability as the final heartbeat frequency estimation result.

In some embodiments of the present disclosure, the step of extracting reserved heartbeat frequency set from the heartbeat frequency candidates includes: comparing each heartbeat frequency candidate with the heartbeat frequency candidates obtained in the past within the second preset time; eliminating the heartbeat frequency candidate if the gap to the heartbeat frequency candidates in the past within the second preset time is greater than the third preset threshold; joining the frequency into the reserved heartbeat frequency set if the gap between the frequency and the heartbeat frequency candidates in the past within the second preset time is less than the third preset threshold.

According to another aspect of an embodiment of the present disclosure, a signal processing apparatus is further provided, which includes: the processing module, which is configured to extract the chest cavity movement signal from the radar echoes that are the electromagnetic signals emitted by radar and reflected from the target; the filtering module, which is configured to obtain the respiratory signal and the heartbeat signal by performing filter process on the chest cavity movement signal; and the signal processing module, which is configured to obtain the respiratory frequency by performing joint time-frequency processing on the respiratory signal, and obtain the heartbeat frequency by performing joint time-frequency processing and statistical analysis on the heartbeat signal.

The embodiment of the present disclosure further provides a processor, which is configured to operate the program. The signal processing method described above is performed when the program is operated.

The embodiment of the present disclosure further provides a computer storage medium, which is configured to store the software program that used to control the device where the computer storage medium located and perform any signal processing method described above.

In the embodiments of the present disclosure, the chest cavity movement signal is extracted from the radar echoes, which are the electromagnetic signals emitted by radar and reflected from the target; the respiratory signal and the heartbeat signal are obtained by performing filter process on the chest cavity movement signal; the respiratory frequency is obtained by performing joint time-frequency processing on the respiratory signal, and the heartbeat frequency is obtained by performing joint time-frequency processing and statistical analysis on the heartbeat signal. By performing joint time-frequency analysis on the respiratory signal and the heartbeat signal respectively, the purpose of determining the respiratory frequency and the heartbeat frequency in the radar echo signal effectively and improving the estimation accuracy and stability of the method is achieved. Therefore, the accuracy of the respiratory frequency and the heartbeat frequency estimation result is improved. And the technical problem of low estimation accuracy of the respiratory frequency and heartbeat frequency, which is induced by only utilizing the time domain characteristics or frequency-domain characteristics of a radar signal during analysis in the related arts that detecting heartbeats and respiration by radar, is resolved.

### BRIEF DESCRIPTION OF THE DRAWINGS

The accompanying drawings described herein are used to provide a further understanding of the present disclosure, and constitute a part of this application. The exemplary embodiments of the present disclosure and the description thereof are used to explain the present disclosure, but do not constitute improper limitations to the present disclosure. In the drawings:

FIG. 1 is a flowchart of a signal processing method according to an embodiment of the present disclosure.

FIG. 2 is a schematic diagram of a main process for signal processing according to an implementation of the present disclosure.

FIG. 3 is a flowchart of a signal processing method according to an implementation of the present disclosure.

FIG. 4 is a schematic diagram of a signal processing apparatus according to an embodiment of the present disclosure.

### DETAILED DESCRIPTION OF THE EMBODIMENTS

In order to enable those skilled in the art to better understand the solutions of the present disclosure, the technical solutions in the embodiments of the present disclosure will be clearly and completely described below in combination with the drawings in the embodiments of the present disclosure. It is apparent that the described embodiments are only part of the embodiments of the present disclosure, and are not all of the embodiments. Based on the embodiments in the present disclosure, all other embodiments obtained by those ordinary skilled in the art without creative work shall all fall within the protection scope of the present disclosure.

It is to be noted that the terms “first”, “second” and the like in the description, claims and the above mentioned drawings of the present disclosure are used for distinguishing similar objects rather than describing a specific sequence or a precedence order. It should be understood that the data used in such a way may be exchanged where appropriate, so that the embodiments of the present disclosure can be implemented in an order other than those illustrated or described below. In addition, the terms “include” and “have” and any variations thereof are intended to cover non-exclusive inclusions. For example, it is not limited for processes, methods, systems, products or devices containing a series of steps or units to clearly list those steps or units, and other steps or units which are not clearly listed or are inherent to these processes, methods, products or devices may be included instead.

An embodiment of the present invention provides a method embodiment of a signal processing method. It is to be noted that the steps shown in the flow diagram of the accompanying drawings may be executed in a computer system, such as a set of computer-executable instructions, and although a logical sequence is shown in the flow diagram, in some cases, the steps shown or described may be executed in a different order.

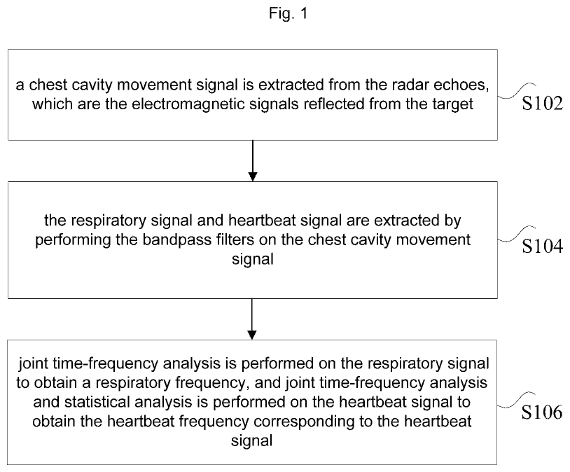

FIG. 1 is a flowchart of a signal processing method according to an embodiment of the present disclosure. As shown in FIG. 1, the method includes the following steps.
At step S102, a chest cavity movement signal is extracted from the radar echoes, which are the electromagnetic signals reflected from the target.
At step S104, the respiratory signal and heartbeat signal are extracted by performing the bandpass filters on the chest cavity movement signal, wherein the respiratory signal corresponds to a respiratory bandpass frequency of the filter, and the heartbeat signal corresponds to a heartbeat bandpass frequency of the filter.
At step S106, joint time-frequency analysis is performed on the respiratory signal to obtain a respiratory frequency, and joint time-frequency analysis and statistical analysis is performed on the heartbeat signal to obtain the heartbeat frequency corresponding to the heartbeat signal.

Through the above steps, the chest cavity movement signal is extracted from the radar echoes, which are the electromagnetic signals emitted by radar and reflected from the target; the respiratory signal and the heartbeat signal are extracted by performing filter process on the chest cavity movement signal; the respiratory frequency is obtained by performing joint time-frequency processing on the respiratory signal, and the heartbeat frequency is obtained by performing joint time-frequency processing and statistical analysis on the heartbeat signal. By performing joint time-frequency analysis on the respiratory signal and the heartbeat signal respectively, the purpose of determining the respiratory frequency and the heartbeat frequency in the radar echo signal effectively and improving the performance on accuracy and stability is achieved. Therefore, the accuracy of the respiratory frequency and the heartbeat frequency estimation result is improved. And the technical problem of low estimation accuracy of the respiratory frequency and heartbeat frequency, which is induced by only utilizing the time domain characteristics or frequency-domain characteristics of a radar signal during analysis in the related arts that detecting heartbeats and respiration by radar, is resolved.

The radar echo signal is an echo generated by the electromagnetic wave emitted by radar and reflected by the target object. The target object may be a person. And the radar may be pulse radar or continuous wave radar.

The above-mentioned processing of a radar echo signal to obtain a chest cavity movement signal may screen a signal portion with high energy from the radar echo signal as the chest cavity movement signal to facilitate the following filtering and arithmetic processing. It may further be an operation of modifying the radar echo signal, such as denoising, to enhance the accuracy and efficiency of the radar echo signal, so as to improve the accuracy of signal processing.

The filter is used to extract the respiratory signal and the heartbeat signal from the chest cavity movement signal. The radar echo signal is a superimposed signal of the respiratory signal and the heartbeat signal of the target object. For facilitating processing and analysis, the filters are used to separate the respiratory signal and the heartbeat signal from the chest cavity movement signal.

Specifically, the respiratory signal can be extracted by performing the filter, of which the passband is setting as the respiratory bandpass frequency, on the chest cavity movement signal. The heartbeat signal can be extracted by performing the filter, of which the passband is setting as the heartbeat bandpass frequency, on the chest cavity movement signal. Therefore, the respiratory signal and the heartbeat signal in the chest cavity movement signal can be effectively separated.

Joint time-frequency domain processing is performed on the respiratory signal to obtain the respiratory frequency corresponding to the respiratory signal, and joint time-frequency domain processing is performed on the heartbeat signal to obtain the heartbeat frequency corresponding to the heartbeat signal. Compared with the related art that only time domain analysis or frequency-domain analysis is performed on the signals, the present disclosure is more accurate, and the effectiveness and accuracy of the respiratory frequency and the heartbeat frequency is improved. The technical problem of low estimation accuracy, which is induced by only relies on the time domain characteristics or frequency-domain characteristics of a radar signal during analysis, in the related technologies of radar based heartbeat frequency and respiration frequency estimation method is solved.

In some embodiments of the present disclosure, the step of extracting the chest cavity movement signal from the radar echoes includes: performing Fourier transform on each modulation signal from the radar echoes along time axis to obtain the range profiles corresponding to each linear modulation signal; extracting the signals from the range bins with a maximum magnitude along the range axis in the range profiles and arranging the signals according to the time axis to obtain the respiratory-heartbeat signal; and performing unwrapping process on the respiratory-heartbeat signal to obtain the chest cavity movement signal.

The step of processing the radar echo signal may be performed as following:

(1) Fourier transform is performed on the received radar chirp signals to obtain the range profile range(r), that is, the above-mentioned process of performing Fourier transform on the chirp signals from the radar echoes along fast time axis to obtain the range profiles.

range(r)=∫_{-∞}^{+∞}chirp(τ)e^{-jrt}dτ,

where, chirp(τ) is a received radar chirp signal, r is the range bins that represent the distance between the target and radar, j is the imaginary sign, and τ is the integration variable.

(2) The vital sign signal of the chest wall of human body at the current moment is extracted from the range bins with the maximum magnitude in range(r).

P=max(abs(range(r))),

R=find(abs(range(r))==P),

data=range(R),

where, abs( ) represent to take an absolute value, max( ) represent to obtain a maximum value, P is the maximum value in range profile range(r), and find( ) represent to obtain the index of the signal that the energy is P.

The main purpose of this step is to extract the slow-time signals with the maximum power in range profiles.

(3) The signal ‘data’ is obtained by performing the above steps (1) and (2) on each received radar chirp signal, and the respiratory-heartbeat signal is obtained by collecting consecutive signal ‘data’ together.

signal(t)=[data1 data2 . . . datan],

where, data1 . . . datan represent ‘data’ obtained in the chirp signal at moment 1 to moment n.

(4) Extract the phase signal phase_{signal}(t) from signal(t), and performing phase unwrapping procedure on it to obtain the signal phase_{data}(t).

phase_{signal}(t)=angle(signal(t)),

phase_{data}(t)=unwrap(phase_{signal}(t),

where, angle( ) represents to extract the phase signal, and unwrap( ) means the phase unwrapping procedure. Phase unwrapping is to regularize the phase signal, which is a well-known and public technology in the technical field.

In some embodiments of the present disclosure, the step of performing the filters on the chest cavity movement signal to obtain the respiratory signal and the heartbeat signal includes: setting the passband of the filter as the respiratory bandpass frequency, and performing the filter on the chest cavity movement signal to obtain the respiratory signal; and setting the passband of the filter as the heartbeat bandpass frequency, and performing the filter on the chest cavity movement signal to obtain the heartbeat signal.

Since a respiratory frequency of a human body is usually in a range of 0.1 to 0.8 Hz, in this embodiment, the respiratory bandpass frequency can be set as 0.1 to 0.8 Hz. Since a heartbeat frequency of the human body is usually in a range of 0.7 to 2.5 Hz, in this embodiment, the heartbeat bandpass frequency can be set as 0.7 to 2.5 Hz. Therefore, the heartbeat signal and the respiratory signal are separated from the radar echo signal.

In some embodiments of the present disclosure, the step of performing joint time-frequency processing on the respiratory signal to obtain the respiratory frequency includes: determining the effective number of respiratory periods of the respiratory signal in time domain; determining the effective respiratory frequency estimated in time domain according to the effective number of respiratory periods and the duration of the respiratory signal; performing Fourier transform on the respiratory signal to obtain the frequency spectrum in frequency domain; and determining the respiratory frequency corresponding to the respiratory signal according to the effective respiratory frequency estimated in time domain and the frequency spectrum of the respiratory signal in frequency domain.

In time domain, the effective number of respiratory periods of the respiratory signal and the effective frequency of respiratory are extracted. In frequency domain, the respiratory frequency corresponding to the respiratory signal is determined according to the effective frequency of respiratory estimated in time domain and the spectrum of respiratory in frequency-domain. Since the respiratory frequency corresponding to the respiratory signal is obtained by analyzing the respiratory signal in time domain and frequency domain, the estimated result is more accurate compared to the related art that only performs time domain analysis or frequency-domain analysis on the signals. Therefore, the estimation accuracy of the respiratory frequency is improved. And the technical problem of low estimation accuracy, which is induced by only relies on the time domain characteristics or frequency-domain characteristics of a radar signal during analysis, in the related technologies of radar based heartbeat frequency and respiration frequency estimation method is solved.

In some embodiments of the present disclosure, the step of determining the effective number of respiratory periods of the respiratory signal in time domain includes: obtaining the first peak position sequence, that is, the position of peaks with the value is greater than 0, and the first valley position sequence, that is, the position of valleys with the value is less than 0, by traversing the respiration signal in time domain; eliminating peaks, of which distance is less than the first preset threshold, from the first peak position sequence and setting the number of remaining peaks as the number of first effective peaks, and eliminating valleys, of which distance is less than the first preset threshold, from the first valley position sequence and setting the number of remaining valleys as the number of first effective valleys; and determining the effective number of respiratory periods according to the number of first effective peaks and the number of first effective valleys.

In time domain, the first peak position sequence and the first valley position sequence of the respiratory signal are obtained. The fake peaks and valleys are eliminated by using the first preset threshold to screen the first peak position sequence and the first valley position sequence, so that the number of first effective valleys and the number of first effective valleys are obtained. The effective number of respiratory periods is determined according to the number of first effective peaks and the number of first effective valleys. By distinguishing whether the distance between the adjacent peaks meets the first preset threshold, it can be determined whether the peaks are caused by noise or clutter, and the number of effective periods of the respiratory signal can be obtained.

In some embodiments of the present disclosure, the step of determining the respiratory frequency corresponding to the respiratory signal according to the effective respiratory frequency estimated in time domain and the frequency spectrum of the respiratory signal in frequency domain includes: determining the effective respiratory frequency range according to the effective respiratory frequency estimated in time domain and the allowable respiratory frequency value; selecting the frequency point with the maximum magnitude within the effective respiratory frequency range from the spectrum of the respiratory in frequency domain, and determining the frequency with the maximum spectral energy as the respiratory frequency.

The allowable respiratory frequency value and the effective respiratory frequency may form the effective frequency range of the respiratory signal. Specifically, if the duration of the signal is T, and the effective number of respiratory periods is N, as a result, the effective respiratory frequency is N/T. The allowable respiratory frequency value may be 0.1 Hz, then the effective respiratory frequency range is [0.1 Hz, N/T+0.1 Hz]. The frequency in the respiratory signal that falls within the effective respiratory frequency range is likely to be the real respiratory frequency. The frequency with the maximum power in frequency domain is selected to be the final respiratory frequency. Therefore, joint time-frequency domain processing is performed according to the respiratory signal to obtain the final respiratory frequency.

Assuming that the number of effective respiratory periods is N and the duration of the respiratory signal is T, so that, an upper limitation frequency of the respiratory frequency is obtained as N/T. Since the noise may bring in extra peaks and valleys, the number of effective respiratory period that estimated by counting the number of peaks and valleys, which is susceptible to noise, may be greater than the number of actual respiratory periods. The spectrum of respiratory is obtained by performing Fourier transform on the respiratory signal. Take the estimated result N/T as the upper bound and extract the frequency with the maximum spectrogram power as the respiratory frequency. In the above procedure, a frequency range is determined by the estimation procedure in time domain, and an accurate estimation result is obtained by the estimation procedure in frequency-domain.

In some embodiments of the present disclosure, the step of performing joint time-frequency domain processing and statistical analysis of data on the heartbeat signal to obtain a heartbeat frequency corresponding to the heartbeat signal includes: determining the effective number of heartbeat periods of the heartbeat signal in time domain; estimating the effective frequency of the heartbeat signal in time domain according to the effective number of heartbeat periods and the duration of the heartbeat signal; performing Fourier transform on the heartbeat signal to obtain the spectrum of the heartbeat signal in frequency domain; and estimating the heartbeat frequency of the heartbeat signal according to the effective frequency estimated in time domain and the spectrum distribution of the heartbeat signal in frequency domain.

In time domain, the effective number of heartbeat periods of the heartbeat signal and the effective heartbeat frequency are determined. In the frequency domain, the heartbeat frequency corresponding to the heartbeat signal is determined according to the effective heartbeat frequency and the spectrum of heartbeat signal in frequency-domain. Since the heartbeat frequency corresponding to the heartbeat signal is obtained by the analyzation in time domain and frequency domain, the present disclosure is more accurate compared to the related art that only performs time domain analysis or frequency-domain analysis on the heartbeat signals. Therefore, the analyzation of the radar echo signal is more refined, and the estimation accuracy of the respiratory frequency and the heartbeat frequency is improved. And the technical problem of low estimation accuracy, which is induced by only relies on the time domain characteristics or frequency-domain characteristics of a radar signal during analysis, in the related technologies of radar based heartbeat frequency and respiration frequency estimation method is solved.

In some embodiments of the present disclosure, before determining the effective number of heartbeat periods of the heartbeat signal in time domain of the heartbeat signal, the method further includes: performing normalization process on the heartbeat signal to normalize the variance of the heartbeat signal; and determining the effective number of heartbeat periods of the heartbeat signal in time domain according to the normalized heartbeat signal.

It is to be noted that, since the passband of respiratory frequency is in the range of 0.1 to 0.8 Hz, and the passband of the heartbeat frequency is in the range of 0.7 to 2.5 Hz, the frequency of the respiratory signal and the frequency of the heartbeat signal are partially overlapped. In this embodiment, before determining the effective number of heartbeat periods of the heartbeat signal in time domain, variance normalization process is performed on the heartbeat signal. For example, intercepting the signal by a sliding window with a duration of 1 s and normalizing the variance of the signal in the window ensures that the variance of the processed signal remains at a similar magnitude. Therefore, the impact of a rising edge and a falling edge of the respiratory signal on the waveform of the heartbeat signal is eliminated.

In some embodiments of the present disclosure, the step of determining the effective number of heartbeat periods of the heartbeat signal in time domain includes: obtaining the second peak position sequence and the second valley position sequence, wherein the second peak position sequence is the sequence of the positions of peaks greater than 0, and the second valley position sequence is the sequence of the positions of valleys less than 0, respectively, by traversing the heartbeat signal; eliminating peaks, of which the distance is less than the second preset threshold, from the second peak position sequence and setting the number of remaining peaks as the number of second effective peaks, and eliminating valleys, of which distance is less than the second preset threshold, from the second valley position sequence and setting the number of remaining peaks as the number of second effective valleys; and determining the effectively number of heartbeat periods according to the number of second effective peaks and the number of second effective valleys.

In time domain, the second peak position sequence and the second valley position sequence of the heartbeat signal are obtained. The second peak position sequence and the second valley position sequence are compared with the second preset threshold to eliminate the false peaks and valleys in the heartbeat signal, so that the number of second effective peaks and the number of second effective valleys are obtained. The effective number of heartbeat periods is determined according to the number of second effective peaks and the number of second effective valleys. By distinguishing whether the distance between the adjacent peaks meets the second preset threshold, it can be determined whether the peaks are generated by noise or clutter, and the effective number of heartbeat periods of the heartbeat signal can be obtained.

In some embodiments of the present disclosure, the step of determining the heartbeat frequency corresponding to the heartbeat signal according to the effective frequency of the heartbeat signal in time domain and the spectrum of the heartbeat signal includes: determining the effective heartbeat frequency range according to the effective frequency of the heartbeat signal in time domain and the allowable heartbeat frequency value; extracting all the peaks of the spectrum of the heartbeat signal within the interval of the effective heartbeat frequency range, and recording these peaks as heartbeat frequency candidates; extracting the reserved heartbeat frequency set from the heartbeat frequency candidates; performing maximum likelihood estimation (MLE) on the reserved heartbeat frequencies to determine the probabilities of being the heartbeat frequency for all the frequencies in the reserved heartbeat frequency; modifying the probability of the frequencies in the reserved heartbeat frequency of being the heartbeat frequency via the analysis of historical data, and determining the frequency in the reserved heartbeat frequency with the maximum probability as the heartbeat frequency.

According to the allowable heartbeat frequency value and the effective heartbeat frequency, the effective frequency range of the heartbeat signal can be obtained. Specifically, if the duration of the signal is T, and the effective number of heartbeat periods is Nₕ, so that, the effective heartbeat frequency is Nₕ/T. The allowable heartbeat frequency value may be 0.7 Hz, then the effective frequency range is [0.7 Hz, Nₕ/T+0.1 Hz]. The frequency in the heartbeat signal that falls within the effective heartbeat frequency range is likely to be the real heartbeat frequency. According to the frequency obtained in time domain, the value range of the heartbeat frequency is narrowed, the influence of noise can be reduced to some extent, and the estimation accuracy of heartbeat frequency is improved.

In some embodiments of the present disclosure, the step of performing MLE on the reserved heartbeat frequency set, and determining the probability of the frequencies in the reserved heartbeat frequency of being the heartbeat frequency for all the frequencies in the reserved heartbeat frequency set includes: determining the joint probability density function of the heartbeat signal according to the signal model of the heartbeat signal; determining the optimization problem of parameters including the frequency, the amplitude and the initial phase of the heartbeat signal according to the joint probability density function; obtaining the probability of each frequency in the reserved heartbeat frequency set of being the real heartbeat frequency by solving the optimization problem.

A specific step of MLE includes the following:

Assume that the heartbeat waveform is a sinusoidal signal, and the heartbeat signal can be written in a discrete form expressed as:

x[n]=A cos(2πfn+ϕ)+w[n], n=0,1, . . . ,N−1 (1)

where, x[n] is the noise-corrupted heartbeat signal, A is the amplitude of pure heartbeat signal, f is the heartbeat frequency, ϕ is the initial phase and w[n] is the phase noise. Here, the noise is assumed to be the white gaussian noise with zero mean and variance of σ².

Since the noise is the white gaussian noise, a likelihood function of the heartbeat signal x[n] can be expressed as:

$$p ( x ; n ) = \frac{1}{{( 2 π {σ}^{2} )}^{N / 2}} exp \{ - \frac{1}{2 {σ}^{2}} {\sum }_{n = 0}^{N - 1} {( x [ n ] - A cos ( 2 π fn + ϕ ) )}^{2} \} \qquad ( 2 )$$

In this case, the parameter with the maximum likelihood probability needs to be found, that is, the desired parameters could be obtained by solving the following optimization problem:

$$[ \hat{A} , \hat{f} , \hat{ϕ} ] = {arg max}_{A , f , ϕ} p ( x ; n ) \qquad ( 3 )$$

Since π and σ² are constants, the optimization problem in (3) can be reformulated as the following form:

$$[ \hat{A} , \hat{f} , \hat{ϕ} ] = {arg min}_{A , f , ϕ} J ( A , f , ϕ ) = {\sum }_{n = 0}^{N - 1} {( x [ n ] - A cos ( 2 π fn + ϕ ) )}^{2} \qquad ( 4 )$$

Here, the formula in (5) is defined and is used to rewrite the formula (4).

$$\{ \begin{matrix}{α}_{1} = A cos ϕ \\ \\ {α}_{2} = A sin ϕ\end{matrix} \qquad ( 5 )$$

Now, the optimization problem in (4) can be written as follows:

J(A,f,ϕ)=J(α₁,α₂,f)=Σₙ₌₀^{N-1}(x[n]−α₁ cos(2πfn)−α₂ sin(2πfn))² (6)

Define

$$\{ \begin{matrix}c = {[ 1 , cos ( 2 π f ) , cos ( 4 π f ) , cos ( 6 π fn ) , … , cos ( 2 π f ( N - 1 ) ) ]}^{T} \\ s = {[ 0 , sin ( 2 π f ) , sin ( 4 π f ) , sin ( 6 π fn ) , … , sin ( 2 π f ( N - 1 ) ) ]}^{T}\end{matrix} ,$$

and the optimization problem in (6) can be written as follows:

J(α₁,α₂,f)=(x−α₁c−α₂s)^{T}(x−α₁c−α₂s) (7)

To simplify the derivation, we define

$$\{ \begin{matrix}H = [ c , s ] \\ α = {[ {α}_{1} , {α}_{2} ]}^{T}\end{matrix} ,$$

and J(α₁, α₂, f) in (7) can be reformulated as follows:

J(α₁,α₂,f)=(x−Hα)^{T}(x−Hα) (8)

When the partial derivative of the likelihood J(α₁, α₂, f) with respect to a equals zero, the following equation is obtained:

$$\frac{\partial J ( {α}_{1} {α}_{2} , f )}{\partial α} = 0 ⇒ \hat{α} = {( {H}^{T} H )}^{- 1} {H}^{T} x \qquad ( 9 )$$

Substituting (8) into (9), it can be acquired:

J(α₁,α₂,f)=x^{T}(1−H(H^{T}H)⁻¹H^{T})x (10)

Now, the optimization problem may be written as follows:

min J(A,f,ϕ)⇒max{x^{T}H(H^{T}H)⁻¹H^{T}x} (11)

The optimization problem in (11) can be reformulated as:

$$max \{ {x}^{T} {H ( {H}^{T} H )}^{- 1} {H}^{T} x \} = max \frac{2}{N} {\vert {\sum }_{n = 0}^{N - 1} x [ n ] exp ( - j 2 π fn ) \vert}^{2} \qquad ( 12 )$$

Then, the solution of (3) can be obtained as follows:

$$\hat{f} = {argmax}_{f} \frac{2}{N} {\vert {\sum }_{n = 0}^{N - 1} x [ n ] exp ( - j 2 π fn ) \vert}^{2}$$

$$\hat{A} = \frac{2}{N} \vert {\sum }_{n = 0}^{N - 1} x [ n ] exp ( - j 2 π \hat{f} n ) \vert$$

$$\hat{ϕ} = arctan ( \frac{{\sum }_{n = 0}^{N - 1} x [ n ] sin ( - j 2 π \hat{f} n )}{{\sum }_{n = 0}^{N - 1} x [ n ] cos ( - j 2 π \hat{f} n )} )$$

The probabilities of being the real heartbeat frequency for all the frequencies in the reserved heartbeat frequencies can be evaluated.

In some embodiments of the present disclosure, the step of modifying the probability of the reserved heartbeat frequencies by historical data, and determining a heartbeat frequency of the reserved heartbeat frequency with the maximum probability as the heartbeat frequency includes: determining the historical heartbeat frequencies, which are the estimation results of the heartbeat signal in the past within the first preset time; performing cluster process on the historical heartbeat frequencies while taking each frequency in the reserved heartbeat frequency as the centroid, and obtaining the evaluation result of each frequency, which is the ratio of the number of the historical heartbeat frequencies that are clustered as the class of each centroid to the total number; modifying the probability of the frequency in the reserved heartbeat frequency set of being the real heartbeat frequency by multiplying the ratio obtained from the cluster process; and taking the frequency in the reserved heartbeat frequency set with the maximum probability as the final heartbeat frequency estimation result.

The evaluation result obtained from the MLE process is modified by statistical information of historical data. Statistical analysis is performed on historical heartbeat frequencies obtained in the pass within the first preset time (for example, 30 s). Clustering is carried out with the reserved heartbeat frequencies are used as centers, and the number of historical frequencies assigned to each center is defined as the evaluation result of the historical data for the reserved heartbeat frequencies. Considering the evaluation results provided by MLE analysis process and historical data analysis process comprehensively, the frequency in the reserved heartbeat frequencies obtaining the best evaluation is selected to be the estimation result of heartbeat rate.

In some embodiments of the present disclosure, the step of extracting the reserved heartbeat frequency set from the heartbeat frequency candidates includes: comparing each frequency from the heartbeat frequency candidates with the heartbeat frequency candidates obtained in the past within the second preset time; eliminating the frequency if the gap to the heartbeat frequency candidates in the past within the second preset time is greater than the third preset threshold; and extracting the frequency as the reserved heartbeat frequency if the gap between the frequency and the heartbeat frequency candidates in the past within the second preset time is less than the third preset threshold.

The reserved heartbeat frequencies are extracted from the heartbeat frequency candidates. Each heartbeat frequency candidate is compared with the heartbeat frequency candidates in the past within the second preset time (for example, 5 s). If there is a large distance between the heartbeat frequency candidate and the heartbeat frequency candidates in the past, that is, if the gap between the heartbeat frequency candidate and the heartbeat frequency candidates in the past is larger than the third preset threshold, the frequency is eliminated. If the distance between the heartbeat frequency candidate and the heartbeat frequency candidates in the past within the second preset time does not exceed the third preset threshold, the frequency is extracted as the reserved heartbeat frequency.

For the heartbeat signal, the heartbeat frequency usually changes smoothly and continuously, and there should no abrupt changes under the resting circumstance. Comparing the heartbeat frequency candidate with heartbeat frequency candidates in the past, if the gap is small, which indicates that the heartbeat frequency candidate meets the continuity condition, the frequency is likely to be real heartbeat frequency. If the difference is too large, which means the heartbeat frequency candidate does not meet the continuity condition and may be caused by the noise, the frequency should to be eliminated.

It is to be noted that, an embodiment of this application further provides an implementation in some embodiments of the present disclosure. The implementation is described in detail below.

The implementation provides a respiratory-heartbeat frequency estimation method based on the MLE theory and data statistical analysis, which belongs to the technical field of radar target recognition.

The implementation is intended to provide the respiratory-heartbeat frequency estimation method based the an MLE theory and data statistical analysis. In view of non-contact monitoring approaches of respiration and heartbeat, the respiratory signal and the heartbeat signal are separated by the bandpass filters. Then, respiratory frequency and heartbeat frequency are obtained via the methods such as joint time-frequency domain analysis, estimation theory, and statistical analysis.

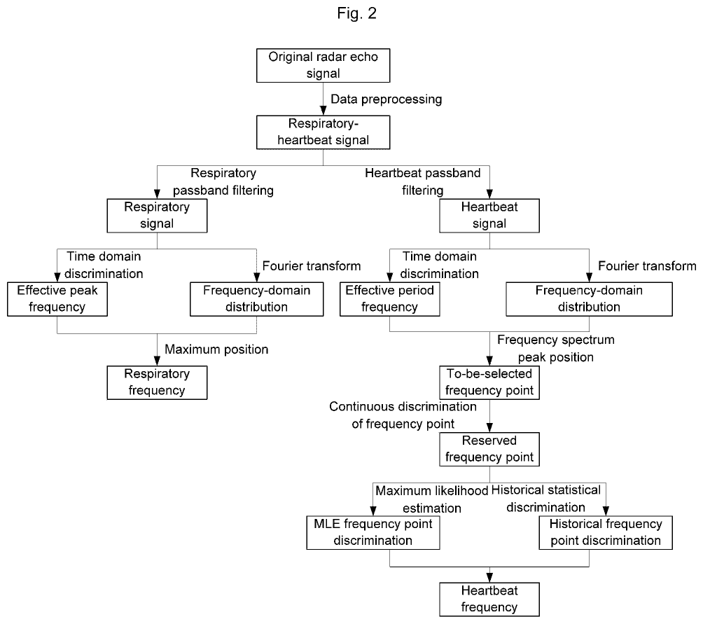

FIG. 2 is a schematic diagram of a main process for signal processing according to an implementation of the present disclosure. As shown in FIG. 2, a respiratory-heartbeat monitoring method based on the estimation theory provided in this implementation mainly includes the data pre-processing process, the respiration frequency estimation process and the heartbeat frequency estimation process. The data pre-processing process includes the following steps:

(1) Fourier transform is performed on each chirp (the linear frequency modulation signal) of the received radar echo to obtain the range profile range (r) of the echo signal.

range(r)=∫_{-∞}^{+∞}chirp(τ)e^{-jrt}dτ

where, chirp(τ) is a received radar chirp signal, r is the range bins that represent the distance between the target and radar, j is the imaginary sign, and τ is the integration variable.

(2) The vital sign signal of the chest wall of human body at the current moment is extracted from the range bins with the maximum magnitude in range(r).

P=max(abs(range(r)))

R=find(abs(range(r))==P)

data=range(R)

where, abs( ) represent to take the absolute value, max( ) represent to obtain the maximum value, P is the maximum value in range profile range(r), and find( ) represent to obtain the index of the signal that the energy is P.

(3) The signal ‘data’ is obtained by performing the above steps (1) and (2) on each received radar chirp signal, and the respiratory-heartbeat signal is obtained by collecting consecutive signal ‘data’ together.

signal(t)=[data1 data2 . . . datan]

where, data1 . . . datan represent ‘data’ obtained in the chirp signal at moment 1 to moment n.

(4) Extract the phase signal phase_{signal}(t) from signal(t), and perform phase unwrapping procedure on it to obtain the signal phase_{data}(t).

phase_{signal}(t)=angle(signal(t))

phase_{data}(t)=unwrap(phase_{signal}(t)

where, angle( ) represents to extract the phase signal, which may be implemented by an arctan function, and unwrap( ) represents to phase unwrapping procedure.

The respiration frequency estimation section includes the following steps:

(1) Perform bandpass filter on the signal phase_{data}(t). Since the respiratory frequency is usually in the range of 0.1 to 0.8 Hz, it is set as the passband of the filter, and the filtered signal is defined as data_{breath}(t).

data_{breath}(t)=filter(phase_{data}(t),bp)

where, filter( ) is the filter, and bp is a passband range of the filter. In this case, the passband range of the respiratory filter is 0.1 to 0.8 Hz.

(2) Determine the number of peaks and valleys of the respiratory signal in time domain. Traverse the signal data_{breath}(t), and extract the peak position with the peak value greater than 0 as the peak position sequence peak_{breath}(n) and the valley position with the valley value less than 0 as the valley position sequence valley_{breath}(n).

(3) Screen the peak position sequence and the valley position sequence, and define the number of effective periods of the respiratory signal as N_{b}. Traverse the peak position sequence peak_{breath}(n), and eliminate the peaks that are too close to each other from the peak position sequence. Define the number of the remaining peaks as the number of effective peaks N1. Similarly, traverse the valley position sequence valley_{breath}(n), eliminate the valleys that are too close to each other from the valley position sequence, and define the number of the remaining valleys as the number of the effective valleys N2. Then the number of effective periods is shown as follows:

N_{b}(N1+N2)/2

(4) Estimate the frequency of the respiratory signal in frequency domain. Assume that the duration of the signal data_{breath}(t) is T, and the effective number of respiratory periods obtained by step (3) is N, so that the effective respiratory frequency is N/T. Performing Fourier transform on the signal data_{breath}(t) to obtain the spectrum of the respiratory signal frequency_{breath}(f), take the effective respiratory frequency as the upper bound and extract the frequency with the maximum magnitude in the interval of [0.1 Hz, N/T+0.1 Hz] as the final respiratory frequency f_{breath}.

The heartbeat frequency estimation section includes the following steps:

(1) Perform bandpass filter on the signal phase_{data}(t). Since the heartbeat frequency is usually in the range of 0.7 to 2.5 Hz, it is set as the passband of the heartbeat filter, and the filtered signal is defined as dataₕₑₐᵣₜ(t).

dataₕₑₐᵣₜ(t)=filter(phase_{data}(t),bp2)

where, bp2 is a passband range of the filter. In this case, the passband range of the heartbeat filter is 0.7 to 2.5 Hz.

(2) Perform normalization process on the heartbeat signal. Intercepting the signal by a sliding window with a duration of is 1 s and normalizing the variance of the signal in the window ensures that the variance of the processed signal remains at a similar magnitude.

(3) Define the number of peaks and valleys of the heartbeat signal in time domain. Traversing the signal dataₕₑₐᵣₜ(t), extract the peak position with the peak value greater than 0 as the peak position sequence peakₕₑₐᵣₜ(n) and the valley position with the valley value less than 0 as the valley position sequence valleyₕₑₐᵣₜ(n).

(4) Extract the number of effective heartbeat periods Nₕ from the peak position sequence and the valley position sequence. Traverse the peak position sequence peakₕₑₐᵣₜ(n), and eliminate the peaks that are too close to each other from the peak position sequence. Define the number of the remaining peaks as N1. Traverse the valley position sequence valleyₕₑₐᵣₜ(n), and eliminate the valleys that are too close to each other from the valley position sequence. Define the number of the remaining valleys as N2. Then the number of effective periods is shown as follows:

Nₕ(N1+N2)/2

(5) Extract the heartbeat frequency candidates from the spectrum of the heartbeat signal in frequency domain. Assume that the duration of the signal dataₕₑₐᵣₜ(t) is T, and the effective number of heartbeat periods obtained by step (4) is Nₕ, so that the effective heartbeat frequency is Nₕ/T. Fourier transform is performed on the signal dataₕₑₐᵣₜ(t) to obtain the spectrum of the heartbeat signal frequencyₕₑₐᵣₜ(f). Taking the effective heartbeat frequency as the upper bound, extract the peaks from the spectrum in the interval of [0.7, Nₕ/T+0.3 Hz] and record them as the heartbeat frequency candidates.

(6) Perform continuity analysis on the heartbeat frequency candidates. Comparing the frequency in heartbeat frequency candidates with the heartbeat frequency candidates in the past within 5 s, if the gap between the frequency from the heartbeat frequency candidates and the heartbeat frequency candidates in the past is too large, abandon it. If the gap between the frequency from the heartbeat frequency candidates and the heartbeat frequency candidates in the past is small, extract it as the reserved heartbeat frequency.

(7) Perform MLE on the reserved heartbeat frequency set. Each reserved heartbeat frequency is evaluated by MLE to determine the possibility of being the heartbeat frequency.

(8) Modify the evaluation obtained from MLE by the statistical information from historical data. Statistical analysis is performed on historical heartbeat frequencies obtained in the past within 30 s. Performing clustering method on the historical frequencies with the reserved heartbeat frequencies are set to be the centroids, the number of historical frequencies assigned to each centroids is a defined as the evaluation result. Considering the evaluation results provided by MLE analysis process and historical data analysis process comprehensively, the frequency in the reserved heartbeat frequencies obtaining the best evaluation is selected to be the estimation result of heartbeat rate.

Different from other non-contact respiratory-heartbeat frequency estimation method, this implementation proposes a unique procedure: firstly, the respiratory signal and the heartbeat signal are separated by bandpass filters; secondly, joint time-frequency domain processing is performed on the respiratory signal and the heartbeat signal; finally, the continuous determination, the MLE analysis process and the historical data analysis process are performed on the heartbeat frequency candidates. Compared with the method in the related art, the estimation result of this implementation is more accurate, while the joint time-frequency domain analysis process can effectively improve the accuracy of the result and the utilization of estimation theory can reduce the impact of noise and clutter effectively.

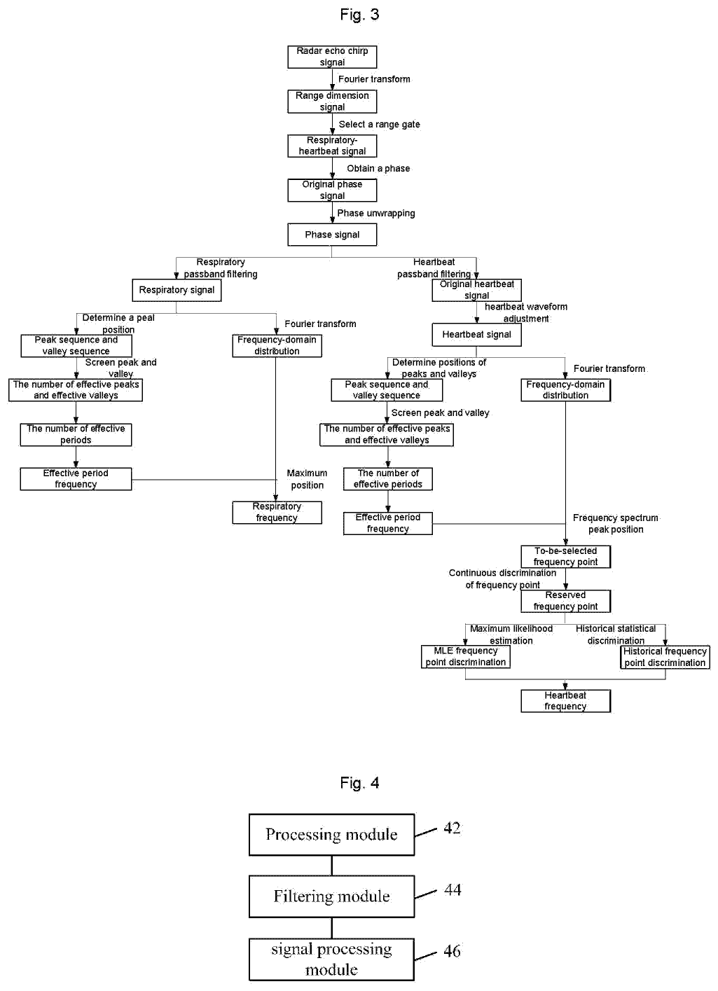

FIG. 3 is a flowchart of a signal processing method according to an implementation of the present disclosure. As shown in FIG. 3, the respiratory-heartbeat frequency estimation method based on the MLE theory and data statistical analysis provided in this implementation includes the pre-processing step, the respiration frequency estimation step and the heartbeat frequency estimation step. The data pre-processing step includes the following:

(1) Perform Fourier transform on each chirp of the received radar echoes to obtain the range profile range (r) of the echo signal.

range(r)=∫_{-∞}^{+∞}chirp(τ)e^{-jrt}dτ

where, chirp(τ) is a received radar chirp signal, r is the range bins that represent the distance between the target and radar, j is the imaginary sign, and τ is the integration variable.

(2) Extract the vital sign signal of the chest wall of human body at the current moment from the range bins with the maximum magnitude in range(r).

P=max(abs(range(r)))

R=find(abs(range(r))==P)

data=range(R)

where, abs( ) represent to take the absolute value, max( ) represent the maximum value, P is the maximum value in range profile range(r), and find( ) represent to obtain the index of the signal that the energy is P.

The purpose of this step is to extract the signal from the range gate with the maximum power in range profile.

(3) The signal ‘data’ is obtained by performing the above steps (1) and (2) on each received radar chirp signal, and the respiratory-heartbeat signal is obtained by collecting consecutive signal ‘data’ together.

signal(t)=[data1 data2 . . . datan]

where, data1 . . . datan represent ‘data’ obtained in the chirp signal at moment 1 to moment n.

(4) Extract the phase signal phase_{signal}(t) from signal(t), and perform phase unwrapping procedure on it to obtain the signal phase_{data}(t).

phase_{signal}(t)=angle(signal(t))

phase_{data}(t)=unwrap(phase_{signal}(t)

where, angle( ) represents to extract the phase signal, and unwrap( ) represents the phase unwrapping procedure. Phase unwrapping is to regularize the phase signal, which is a well-known and public technology in the technical field.

The respiration frequency estimation section includes the following steps:

(1) Perform bandpass filter on the signal phase_{data}(t). Since the respiratory frequency is usually in the range of 0.1 to 0.8 Hz, it is set as the passband of the respiratory filter, and the filtered signal is defined as data_{breath}(t).

data_{breath}(t)=filter(phase_{data}(t),bp)

where, filter( ) is the filter algorithm, and bp is a passband range of the filter. In this case, the passband range of the respiratory filter is 0.1 to 0.8 Hz. The filter algorithm is a well-known and public technology in the technical field.

The purpose of this step is to obtain the signal within the respiratory passband.

(2) Determine the peak position sequence and the valley position sequence of the respiratory signal in time domain. The peak positions with the peak value greater than 0 are extracted as the peak position sequence peak_{breath}(n) and the valley positions with the valley value less than 0 are extracted as the valley position sequence valley_{breath}(n), while traversing the signal data_{breath}(t).

(3) Extract the number of effective periods (N_{b}) of the respiratory signal from the peak position sequence and the valley position sequence. Traversing the peak position sequence peak_{breath}(n), eliminate the peaks, which are too close to each other, from the peak position sequence, and define the number of the remaining peaks as N1. Likewise, traversing the valley position sequence valley_{breath}(n), eliminate the valleys that are too close to each other from the valley position sequence and define the number of the remaining valleys as N2. Then the number of effective periods is obtained as follows:

N_{b}(N1+N2)/2

The purpose of this step is to determine whether the peaks and valleys are generated by noise or clutter via the distance between the adjacent peaks, and to eliminate the peaks and valleys caused by these disturbances.

(4) Estimate the frequency of the respiratory signal in frequency domain. Assume that the duration of the signal data_{breath}(t) is T, and the effective number of respiratory periods obtained by step (3) is N_{b}, so that the effective respiratory frequency is N_{b}/T. Fourier transform is performed on the signal data_{breath}(t) to obtain the spectrum of the respiratory signal frequency_{breath}(f), and the effective respiratory frequency is set as the upper bound and the frequency with the maximum magnitude in the interval of [0.1 Hz, N_{b}/T+0.1 Hz] is extracted as the final respiratory frequency f_{breath}.

Assume that there are N_{b} effective respiratory periods in the duration of T, so that, the upper bound of the respiratory frequency estimated in time domain is N_{b}/T. Since the respiratory signal is susceptible to noise, which would bring in extra peaks and valleys, the estimation result obtained by counting the number of effective respiratory periods should be greater than the real number of respiratory periods. Performing Fourier transform on the respiratory signal can obtain the spectrum of the respiratory signal, take the respiratory frequency estimated in time domain (N_{b}/T) as the upper bound and extract the frequency with the maximum power as the final respiratory frequency.

The method determines a frequency range through the estimation result in time domain, and the final frequency is obtained through the fine estimation process in frequency domain.

The heartbeat frequency estimation section includes the following steps:

(1) Perform bandpass filter on the signal phase_{data}(t). Since the heartbeat frequency is usually in the interval of 0.7 to 2.5 Hz it is set as the passband of the heartbeat filter, and the filtered signal is defined as dataₕₑₐᵣₜ(t).

dataₕₑₐᵣₜ(t)=filter(phase_{data}(t),bp2)

where, bp2 is a passband range of the filter. In this case, the passband range of the heartbeat filter is 0.7 to 2.5 Hz.

The purpose of this step is to obtain the signal within the heartbeat passband.

(2) Perform normalization process on the heartbeat signal. Intercepting the signal by a sliding window with a duration of is 1 s and normalizing the variance of the signal in the window ensures that the variance of the processed signal remains at a similar magnitude.

The purpose of this step is to eliminate the impact of a rising edge and a falling edge of the respiratory signal on the waveform of the heartbeat signal.

(3) Determine the number of peaks and valleys of the heartbeat signal in time domain. Extract the peak position with the peak value greater than 0 as the peak position sequence peakₕₑₐᵣₜ(n) and the valley position with the valley value less than 0 as the valley position sequence valleyₕₑₐᵣₜ(n) from the signal dataₕₑₐᵣₜ(t).

(4) Extract the effective number of heartbeat periods (Nₕ) from the peak position sequence and the valley position sequence. Traversing the peak position sequence peakₕₑₐᵣₜ(n), eliminate the peaks that are too close to each other from the peak position sequence, and define the number of the remaining peaks as N1. Traverse the valley position sequence valleyₕₑₐᵣₜ(n), and eliminate the valleys that are too close to each other from the valley position sequence. The number of the remaining valleys is defined as N2. Then the effective number of heartbeat periods is extracted as follows:

Nₕ(N1+N2)/2

The purpose of this step is to determine whether the peaks and valleys are generated by noise or clutter based on the distance between the adjacent peaks, and to eliminate the peaks and valleys caused by noise.

(5) Extracted the heartbeat frequency candidates from the spectrum of the heartbeat signal in frequency domain. Assume that the duration of the signal dataₕₑₐᵣₜ(t) is T, and the effective number of heartbeat periods obtained by step (4) is Nₕ, so that the effective heartbeat frequency is Nₕ/T. Perform Fourier transform on the signal dataₕₑₐᵣₜ(t) to obtain spectrum frequencyₕₑₐᵣₜ(f) of the heartbeat signal. Taking the effective heartbeat frequency Nₕ/T as the upper bound, extract the peaks from the spectrum in the interval of [0.7, Nₕ/T+0.3 Hz], and record them as the heartbeat frequency candidates.

The purpose of this step is to extract the frequency that are likely to be the real heartbeat frequency from the search region with the estimation result obtained in time domain as the upper bound. In this way, the search region for heartbeat frequency estimation is further reduced, which means that only the noise within the search region has the influence on the estimation result, leading to an increase of the SNR and the estimation accuracy.

(6) Perform continuity analysis on the heartbeat frequency candidates. Comparing the heartbeat frequency candidate with the heartbeat frequency candidates in the past within 5 s, if the gap between the frequency from the heartbeat frequency candidate and the heartbeat frequency candidates in the past is too large, abandon it, and if the gap is small, extract the frequency as the reserved heartbeat frequency.

The purpose of this step is to eliminate the frequency generated by noise. Under the resting circumstance, the heartbeat frequency usually changes continuously over time, that is, the frequency of the heartbeat signal does not change abruptly. Comparing the frequency at the current moment with the heartbeat frequency candidates in the past, if the gap is very small, it indicates that the heartbeat frequency candidate change continuously over time, which means the frequency is likely to be the heartbeat frequency. If the gap is too large, that means the frequency changes abruptly, and it may be caused by the noise, should to be abandoned.

(7) Perform MLE on the reserved heartbeat frequency candidates. Each reserved heartbeat frequency is evaluated by MLE to determine the possibility of being the heartbeat frequency.

The purpose of this step is to evaluate the possibility of being the real heartbeat frequency for each frequency in the reserved heartbeat frequency by MLE analysis.

(8) Modify the evaluation result from MLE analysis process by the statistical information from historical data. Statistical analysis is performed on historical heartbeat frequencies, which is obtained in the past within 30 s. Performing clustering process on the historical frequencies with the reserved heartbeat frequencies are set to be the centers, the number of historical frequencies assigned to each center is defined as the evaluation result. Considering the evaluation results provided by MLE analysis process and historical data analysis process comprehensively, the frequency in the reserved heartbeat frequencies obtaining the best evaluation is selected to be the estimation result of heartbeat rate.

The purpose of this step is to modify the evaluation result of MLE by statistical analysis on the historical data, and select the reserved heartbeat frequency obtaining the beat evaluation, which is obtained by combining the historical data analysis result and the MLE analysis result, to be the final estimation result.

The MLE theory is used during the evaluation of heartbeat. The MLE method is introduced here as follows.

Assuming that the heartbeat signal is a sinusoidal signal, the signal model is shown as follows:

x[n]=A cos(2πfn+ϕ)+w[n], n=0,1, . . . ,N−1 (1)

where, x[n]is the heartbeat signal, A is the amplitude of the heartbeat, f is the heartbeat frequency, ϕ is the initial phase of the heartbeat signal, and w[n] is the noise, which is assumed to be white gaussian noise with a mean value of 0 and variance of σ².

Since the noise is white gaussian noise, the joint probability density function of the heartbeat signal x[n] can be obtained as follows:

$$p ( x ; n ) = \frac{1}{{( 2 π {σ}^{2} )}^{N / 2}} exp \{ - \frac{1}{2 {σ}^{2}} {\sum }_{n = 0}^{N - 1} {( x [ n ] - A cos ( 2 π fn + ϕ ) )}^{2} \} \qquad ( 2 )$$

In this case, the parameter with the maximum probability can be obtained by solving the following optimization problem:

$$[ \hat{A} , \hat{f} , \hat{ϕ} ] = {argmax}_{A , f , ϕ} p ( x ; n ) \qquad ( 3 )$$

Since π and σ² are fixed constants, the optimization problem can be reformulated as:

$$[ \hat{A} , \hat{f} , \hat{ϕ} ] = {argmin}_{A , f , ϕ} J ( A , f , ϕ ) = {\sum }_{n = 0}^{N - 1} {( x [ n ] - A cos ( 2 π fn + ϕ ) )}^{2} \qquad ( 4 )$$

Here, the transformation of the parameters is defined in formula (5), and the expression in (4) can be written as (6).

$$\{ \begin{matrix}{a}_{1} = A cos ϕ \\ \\ {α}_{2} = A sin ϕ\end{matrix} \qquad ( 5 )$$

$$J ( A , f , ϕ ) = J ( {α}_{1} , {α}_{2} , f ) = {\sum }_{n = 0}^{N - 1} {( x [ n ] - {α}_{1} cos ( 2 π fn ) - {α}_{2} sin ( 2 π fn ) )}^{2} \qquad ( 6 )$$

Let

$$\{ \begin{matrix}c = {[ 1 , cos ( 2 π f ) , cos ( 4 π f ) , cos ( 6 π fn ) , … , cos ( 2 π f ( N - 1 ) ) ]}^{T} \\ s = {[ 0 , sin ( 2 π f ) , sin ( 4 πf ) , sin ( 6 π fn ) , … , sin ( 2 π f ( N - 1 ) ) ]}^{T}\end{matrix} ,$$

the optimization problem can be reformulated as follows:

J(α₁,α₂,f)=(x−α₁c−α₂s)^{T}(x−α₁c−α₂s) (7)

$$\{ \begin{matrix}H = [ c , s ] \\ α = {[ {α}_{1} , {α}_{2} ]}^{T}\end{matrix} ,$$

Let the optimization problem can be reformulated as:

J(α₁,α₂,f)=(x−Hα)^{T}(x−Hα) (8)

When the differentiation of the likelihood J(α₁, α₂, f) with respect to α equals 0, we can derive the following equation:

$$\frac{\partial J ( {α}_{1} , {α}_{2} , f )}{\partial α} = 0 ⇒ \hat{α} = {( {H}^{T} H )}^{- 1} {H}^{T} x \qquad ( 9 )$$

Substituting (8) into (9), we can acquire:

J(α₁,α₂,f)=x^{T}(1−H(H^{T}H)⁻¹H^{T})x (10)

In this case, the optimization problem can be written as follows:

min J(A,f,ϕ)⇒max{x^{T}H(H^{T}H)⁻¹H^{T}x} (11)

In this case, the optimization problem is as follows:

$$max \{ {x}^{T} {H ( {H}^{T} H )}^{- 1} {H}^{T} x \} = max \frac{2}{N} {\vert {\sum }_{n = 0}^{N - 1} x [ n ] exp ( - j 2 π fn ) \vert}^{2} \qquad ( 12 )$$

Then the solution of the optimization problem is obtained as follows:

$$\hat{f} = {argmax}_{f} \frac{2}{N} {\vert {\sum }_{n = 0}^{N - 1} x [ n ] exp ( - j 2 π fn ) \vert}^{2}$$

$$\hat{A} = \frac{2}{N} \vert {\sum }_{n = 0}^{N - 1} x [ n ] exp ( - j 2 π \hat{f} n ) \vert$$

$$\hat{ϕ} = arctan ( \frac{{\sum }_{n = 0}^{N - 1} x [ n ] sin ( - j 2 π \hat{f} n )}{{\sum }_{n = 0}^{N - 1} x [ n ] cos ( - j 2 π \hat{f} n )} )$$

The respiratory-heartbeat frequency estimation method based on the MLE theory and data statistical analysis in this implementation belongs to the technical field of radar target recognition. The method includes: obtaining respiratory-heartbeat signals from the original radar echoes through data pre-processing, using the bandpass filters to extract the respiratory signal and the heartbeat signal from the respiratory-heartbeat signals, and performing joint time-frequency domain analysis on the respiratory signal to extract the respiratory frequency; and performing joint time-frequency domain analysis on the heartbeat signal to extract the heartbeat frequency candidates, and extracting the heartbeat frequency from the heartbeat frequency candidates by continuous analysis process, MLE evaluation process and historical statistical analysis. In view of non-contact respiratory-heartbeat frequency estimation method, this method has high accuracy.

FIG. 4 is a schematic diagram of a signal processing apparatus according to an embodiment of the present disclosure. As shown in FIG. 4, another aspect of the embodiments of the present disclosure further provides a signal processing apparatus. The signal processing apparatus includes a processing module 42, a filtering module 44 and a signal processing module 46. The apparatus is described in detail below.

The processing module 42 is configured to extract the chest cavity movement signal from the radar echoes, which are the electromagnetic signals emitted by radar and reflected from the target. The filtering module 44 is connected to the processing module 42, and is configured to obtain the respiratory signal and the heartbeat signal by performing filter process on the chest cavity movement signal. The signal processing module 46 is connected to the filtering module 44, and is configured to obtain the respiratory frequency by performing joint time-frequency processing on the respiratory signal, and obtain the heartbeat frequency by performing joint time-frequency processing and statistical analysis on the heartbeat signal.

Through the above apparatus, the chest cavity movement signal is extracted from the radar echoes, which are the electromagnetic signals emitted by radar and reflected from the target; the respiratory signal and the heartbeat signal are extracted by performing filter process on the chest cavity movement signal; the respiratory frequency is obtained by performing joint time-frequency processing on the respiratory signal, and the heartbeat frequency is obtained by performing joint time-frequency processing and statistical analysis on the heartbeat signal. By performing joint time-frequency analysis on the respiratory signal and the heartbeat signal respectively, the purposes of determining the respiratory frequency and the heartbeat frequency effectively and improving the estimation performance on accuracy and stability are achieved. Therefore, the accuracy of the respiratory frequency and the heartbeat frequency estimation result is improved. And the technical problem of low estimation accuracy of the respiratory frequency and heartbeat frequency, which is induced by only utilizing the information in time domain or frequency-domain of a radar signal during analyzation in the related arts that estimation heartbeat frequency and respiration frequency by radar, is solved.

According to another aspect of an embodiment of the present disclosure, a processor is further provided and configured to operate a program. The signal processing method described in any of the above is performed when the program is operated.

According to another aspect of an embodiment of the present disclosure, a computer storage medium is further provided and stores a stored program. When the program is operated, a device where the computer storage medium located is controlled to perform the signal processing method described in any of the above.

The serial numbers of the foregoing embodiments of the present disclosure are merely for description, and do not represent the superiority or inferiority of the embodiments.

In the above embodiments of the present disclosure, the description of the embodiments has its own emphasis. For parts that are not described in detail in a certain embodiment, reference may be made to related descriptions of other embodiments.

In the several embodiments provided in this application, it should be understood that, the disclosed technical content can be implemented in other ways. The apparatus embodiments described above are merely illustrative. For example, the division of the units may be a logical function division, and there may be other divisions in actual implementation. For example, a plurality of units or components may be combined or integrated into another system, or some features can be ignored, or not implemented.

The units described as separate components may or may not be physically separated. The components displayed as units may or may not be physical units, that is, the components may be located in one place, or may be distributed on the plurality of units. Part or all of the units may be selected according to actual requirements to achieve the purposes of the solutions of this embodiment.

In addition, the functional units in the various embodiments of the present disclosure may be integrated into one processing unit, or each unit may exist alone physically, or two or more than two units may be integrated into one unit. The above integrated unit can be implemented in the form of hardware, or can be implemented in the form of a software functional unit.

If the integrated unit is implemented in the form of the software functional unit and sold or used as an independent product, it can be stored in the computer readable storage medium. Based on this understanding, the technical solutions of the present disclosure or the parts that contribute to the related art, or all or part of the technical solutions can be embodied in the form of a software product. The computer software product is stored in a storage medium, including a plurality of instructions for causing a computer device (which may be a personal computer, a server, or a network device, and the like) to execute all or part of the steps of the method described in the various embodiments of the present disclosure. The foregoing storage medium includes a USB flash disk, a read-only memory (ROM), a random access memory (RAM), and various media that can store program codes, such as a mobile hard disk, a magnetic disk, or an optical disk.

The above description is merely preferred implementations of the present disclosure, and it should be noted that persons of ordinary skill in the art may also make several improvements and refinements without departing from the principle of the present disclosure, and it should be considered that these improvements and refinements shall all fall within the protection scope of the present disclosure.

1. A signal processing method, comprising:
extracting a chest cavity movement signal from radar echoes, which are electromagnetic signals reflected from a target;
extracting a respiratory signal and heartbeat signal by performing bandpass filters on the chest cavity movement signal, wherein the respiratory signal corresponds to a respiratory bandpass frequency of a filter, and the heartbeat signal corresponds to a heartbeat bandpass frequency of a filter; and
performing joint time-frequency analysis on the respiratory signal to obtain a respiratory frequency, and performing joint time-frequency analysis and statistical analysis on the heartbeat signal to obtain a heartbeat frequency corresponding to the heartbeat signal.

2. The method according to claim 1, wherein extracting a chest cavity movement signal from radar echoes comprises:
performing Fourier transform on each modulation signal from the radar echoes along time axis to obtain range profiles;
extracting signals from range bins with a maximum magnitude along the range axis in the range profiles and arranging signals according to the time axis to obtain a respiratory-heartbeat signal; and
performing unwrapping process on the respiratory-heartbeat signal to obtain the chest cavity movement signal.

3. The method according to claim 2, wherein extracting a respiratory signal and heartbeat signal by performing bandpass filters on the chest cavity movement signal comprises:
setting a passband of the filter as the respiratory bandpass frequency, and performing the filter on the chest cavity movement signal to obtain the respiratory signal; and
setting a passband of the filter as the heartbeat bandpass frequency, and performing the filter on the chest cavity movement signal to obtain the heartbeat signal.

4. The method according to claim 1, wherein performing joint time-frequency processing on the respiratory signal to obtain a respiratory frequency comprises:
determining an effective number of respiratory periods of the respiratory signal in time domain;
determining an effective respiratory frequency estimated in time domain according to the effective number of respiratory periods and the duration of the respiratory signal;
performing Fourier transform on the respiratory signal to obtain a frequency spectrum in frequency domain; and
determining the respiratory frequency corresponding to the respiratory signal according to the effective respiratory frequency estimated in time domain and the frequency spectrum of the respiratory signal in frequency domain.

5. The method according to claim 4, wherein determining the effective number of respiratory periods of the respiratory signal in time domain comprises:
obtaining a first peak position sequence and a first valley position sequence, wherein the position of peaks with the value is greater than 0, the position of valleys with the value is less than 0, by traversing the respiration signal in time domain
eliminating peaks of which distance is less than the first preset threshold from the first peak position sequence and setting the number of remaining peaks as the number of first effective peaks, and eliminating valleys of which distance is less than the first preset threshold from the first valley position sequence and setting the number of remaining valleys as the number of first effective valleys; and
determining the effective number of respiratory periods according to the number of first effective peaks and the number of first effective valleys.

6. The method according to claim 4, wherein determining the respiratory frequency corresponding to the respiratory signal according to the effective respiratory frequency estimated in time domain and the frequency spectrum of the respiratory signal in frequency domain comprises:
determining an effective respiratory frequency range according to the effective respiratory frequency estimated in time domain and an allowable respiratory frequency value; and
determining the frequency with the maximum magnitude within the effective respiratory frequency range from the spectrum of the respiratory in frequency domain as the respiratory frequency.

7. The method according to claim 1, wherein performing joint time-frequency analysis and statistical analysis on the heartbeat signal to obtain the heartbeat frequency corresponding to the heartbeat signal comprises:
determining the effective number of heartbeat periods of the heartbeat signal in time domain;
estimating an effective frequency of the heartbeat signal in time domain according to the effective number of heartbeat periods and the duration of the heartbeat signal;
performing Fourier transform on the heartbeat signal to obtain a spectrum of the heartbeat signal in frequency domain; and
estimating the heartbeat frequency of the heartbeat signal according to the effective frequency estimated in time domain and the spectrum of the heartbeat signal in frequency domain.

8. The method according to claim 7, wherein determining the effective number of heartbeat periods of the heartbeat signal in time domain, the method further comprises:
performing normalization process on the heartbeat signal to normalize the variance of the heartbeat signal; and
determining the effective number of heartbeat periods of the heartbeat signal in time domain according to the normalized heartbeat signal.

9. The method according to claim 7, wherein determining the effective number of heartbeat periods of the heartbeat signal in time domain comprises:
obtaining a second peak position sequence and a second valley position sequence, wherein the second peak position sequence is the sequence of the positions of peaks greater than 0, and the second valley position sequence is the sequence of the positions of valleys less than 0, respectively, by traversing the heartbeat signal;
eliminating peaks of which distance is less than a second preset threshold from the second peak position sequence and defining the number of remaining peaks as the number of second effective peaks, and eliminating the valleys of which distance is less than the second preset threshold from the second valley position sequence and defining the number of remaining valleys as the number of second effective valleys; and
determining the effectively number of heartbeat periods according to the number of second effective peaks and the number of second effective valleys.

10. The method according to claim 7, wherein determining the heartbeat frequency corresponding to the heartbeat signal according to the effective frequency of the heartbeat signal in time domain and the spectrum distribution of the heartbeat signal comprises:
determining an effective heartbeat frequency range according to the effective frequency of the heartbeat signal in time domain and an allowable heartbeat frequency value;
extracting all the peaks from the spectrum distribution of the heartbeat signal within the interval of the effective heartbeat frequency range, and defining these peaks as heartbeat frequency candidates;
extracting reserved heartbeat frequency set from the heartbeat frequency candidates;
performing maximum likelihood estimation (MLE) on the reserved heartbeat frequency set to determine the probabilities of being the heartbeat frequency for all the frequencies in the reserved heartbeat frequency set; and
modifying the probability of the frequencies in the reserved heartbeat frequency set of being the heartbeat frequency via the analysis of historical data, and determining the frequency in the reserved heartbeat frequency with the maximum probability as the heartbeat frequency.

11. The method according to claim 10, wherein performing maximum likelihood estimation (MLE) on the reserved heartbeat frequency set to determine the probabilities of being the heartbeat frequency for all the frequencies in the reserved heartbeat frequency set comprises:
determining a joint probability density function of the heartbeat signal according to a signal model of the heartbeat signal;
determining a optimization problem of parameters including the frequency, the amplitude and the initial phase of the heartbeat signal according to the joint probability density function; and
obtaining the probability of each frequency in the reserved heartbeat frequency set of being the real heartbeat frequency by solving the optimization problem.

12. The method according to claim 10, wherein modifying the probability of the frequency in the reserved heartbeat frequency set of being the real heartbeat frequency via the analysis of historical data, and determining the frequency in the reserved heartbeat frequency set with the maximum probability as the heartbeat frequency comprises:
determining historical heartbeat frequencies, which are the estimation results of the heartbeat signal in the past within the first preset time;
performing cluster process on the historical heartbeat frequencies while taking each frequency in the reserved heartbeat frequency set as the centroid, and obtaining the evaluation result of each frequency, which is the ratio of the number of the historical heartbeat frequencies that are clustered with the frequency in the reserved heartbeat frequency set to the total number;
modifying the probability of the frequency in the reserved heartbeat frequency set of being the real heartbeat frequency by multiplying the ratio obtained from the cluster process; and
taking the frequency in the reserved heartbeat frequency set with the maximum probability as the final heartbeat frequency estimation result.

13. The method according to claim 10, wherein extracting reserved heartbeat frequency set from the heartbeat frequency candidates comprises:
comparing each heartbeat frequency candidate with the heartbeat frequency candidates obtained in the past within the second preset time;
eliminating the heartbeat frequency candidate if the gap to the heartbeat frequency candidates in the past within the second preset time is greater than the third preset threshold; and
joining the frequency into the reserved heartbeat frequency set if the gap between the frequency and the heartbeat frequency candidates in the past within the second preset time is less than the third preset threshold.

14. A signal processing apparatus, comprising:
a processing module, configured to extract the chest cavity movement signal from the radar echoes that are the electromagnetic signals emitted by radar and reflected from the target;
a filtering module, configured to obtain the respiratory signal and the heartbeat signal by performing filter process on the chest cavity movement signal; and
a signal processing module, configured to obtain the respiratory frequency by performing joint time-frequency processing on the respiratory signal, and obtain the heartbeat frequency by performing joint time-frequency processing and statistical analysis on the heartbeat signal.

15. A processor, configured to operate a program, wherein the signal processing method according to claim 1 is performed when the program is operated.

16. The method according to claim 1, wherein the radar is pulse radar or continuous wave radar.

17. The method according to claim 1, wherein the processing of a radar echo signal to obtain a chest cavity movement signal comprises:
denoising the radar echo signal to obtain the chest cavity movement signal.

18. The method according to claim 1, wherein the radar echo signal is a superimposed signal of the respiratory signal and the heartbeat signal of the target object.

19. The method according to claim 6, wherein the frequency of the highest frequency point is the frequency of the largest number in the effective respiratory frequency range.

20. The method according to claim 8, wherein performing variance homogenization on the heartbeat signal to obtain a processed heartbeat signal comprises:
calculating the variance of a signal in the window, and windowing the signal to obtain the processed heartbeat signal.
